(19) United States
(12) Patent Application Publication
(10) Pub. No.: US 2026/0033881 A1
(43) Pub. Date: Feb. 5, 2026

(54) CATHETER APPARATUSES AND SYSTEMS FOR PULSED ELECTRIC FIELD ABLATION THERAPY

(71) Applicant: Alpfa Medical, Inc., Menlo Park, CA (US)

(72) Inventors: Raju VISWANATHAN, Palo Alto, CA (US); John MADDEN, Emerald Hills, CA (US)

(73) Assignee: Alpfa Medical, Inc., Menlo Park, CA (US)

(21) Appl. No.: 19/354,755

(22) Filed: Oct. 9, 2025

Related U.S. Application Data
(63) Continuation-in-part of application No. PCT/US2025/023151, filed on Apr. 4, 2025.
(60) Provisional application No. 63/574,637, filed on Apr. 4, 2024.

(51) Int. Cl. A61B 18/00; A61B 90/00

(52) U.S. Cl. CPC A61B 18/00; A61B 90/37; A61B 2018/00077; A61B 2018/00083; A61B 2018/00178; A61B 2018/00184; A61B 2018/00267; A61B 2018/00577; A61B 2090/3782

(57) ABSTRACT

Systems, devices, and methods described herein relate to catheter devices for therapy delivery. In some embodiments, a catheter device includes an expandable electrode and a needle, where the needle can be advanced distal to a distal end of the expandable electrode and inserted into a tissue site. The expandable electrode and the distal end of the needle can be configured to deliver electroporation to the tissue site.

### CROSS-REFERENCE TO RELATED APPLICATIONS

This application is a continuation-in-part of PCT Application No. PCT/US2025/023151 filed Apr. 4, 2025, which claims priority to and the benefit of U.S. Provisional Patent Application No. 63/574,637, filed Apr. 4, 2024, and entitled “CATHETER APPARATUSES AND SYSTEMS FOR PULSED ELECTRIC FIELD ABLATION THERAPY,” the disclosure of each of which is incorporated by reference herein in its entirety.

### TECHNICAL FIELD

This disclosure describes a catheter apparatus and system for delivery of pulsed electric field ablation therapy via interventional access medical procedures.

### BACKGROUND

Pulsed field ablation has emerged as a potentially useful ablation modality that has been investigated in some tumor applications and has recently been found to be fruitful in the context of cardiac ablation for the treatment of cardiac arrhythmias. This non-thermal ablation modality can be tissue selective and minimize collateral damage while also resulting in a post-ablation natural healing process that preserves the extracellular matrix and overall tissue integrity. While devices and waveforms have been previously devised that are appropriate in the cardiac ablation context, there is a need for new devices and tools that may be more appropriate for use in the context of ablation for the treatment of tumors in other types of soft tissue such as, for example, pancreatic tissue.

### SUMMARY

In some embodiments, an apparatus includes an outer shaft defining a first lumen; an inner shaft disposed within the first lumen and extendable relative to the outer shaft, the inner shaft defining a second lumen; and an expandable electrode disposed on a distal end of the inner shaft, the expandable electrode configured to have a conical shape that increases in diameter from a proximal end to a distal end of the expandable electrode. The expandable electrode is configured to automatically transition from a first unexpanded configuration to a second expanded configuration having the conical shape when the expandable electrode is disposed distal to the outer shaft. The apparatus includes a needle disposed within the second lumen and extendable relative to the inner shaft, the needle including a distal tip that is electrically exposed and a section proximal of the distal tip that is electrically insulated, the distal tip of the needle being insertable into a tissue site. The expandable electrode and the distal tip of the needle, when (1) the expandable electrode is disposed distal to the outer shaft and (2) the needle is extended relative to the inner shaft such that the distal tip of the needle is disposed distally of the distal end of the inner shaft, being configured to apply pulsed field ablation to a zone of tissue at the tissue site.

In some embodiments, an apparatus includes an outer shaft defining a first lumen; an inner shaft disposed within the first lumen and extendable relative to the outer shaft, the inner shaft defining a second lumen; an expandable electrode disposed on a distal end of the inner shaft; and a needle disposed within the second lumen and extendable relative to the inner shaft, the needle including a distal tip that is electrically exposed and a section proximal of the distal tip that is electrically insulated, the distal tip of the needle configured to be advanced distal to the expandable electrode and inserted into the tissue site. The outer shaft and the inner shaft define an annular space therebetween that is configured to deliver a conductive fluid to a region surrounding the expandable electrode. The expandable electrode and the distal tip of the needle, when the needle is inserted into the tissue site and the conductive fluid surrounds the expandable electrode, configured to apply pulsed field ablation via a conductive path established by the conductive fluid to a zone of tissue at the tissue site.

In some embodiments, a method includes positioning a distal portion of a catheter system near a tissue site, the catheter system including an outer shaft defining a first lumen and an inner shaft disposed within the first lumen of the outer shaft; disposing a distal end of the inner shaft distal to the outer shaft such that an expandable electrode disposed at the distal end of the inner shaft is transitioned from an unexpanded state to an expanded state; extending a distal tip of a needle slidably disposed within a second lumen of the inner shaft distal to a distal end of the expandable electrode to insert the needle into the tissue site, the distal tip of the needle being electrically exposed; and applying pulsed field ablation to a zone of tissue at the tissue site while the distal tip of the needle is inserted into the tissue site and the expandable electrode is spaced from the tissue site.

### BRIEF DESCRIPTION OF THE DRAWINGS

FIG. 1 illustrates a catheter system of the present disclosure, according to embodiments.

FIG. 2 illustrates a catheter with a needle and a basket electrode, according to embodiments.

FIG. 3 illustrates a basket electrode of a catheter system, according to embodiments.

FIG. 4 illustrates a catheter device of the present disclosure passing through a channel of an endoscope, according to embodiments.

FIG. 5 illustrates a catheter system of the present disclosure where an outer sheath is included as part of the catheter system, according to embodiments.

FIG. 6 illustrates a catheter system of the present disclosure where an outer sheath is included as part of the catheter system, according to embodiments.

FIG. 7 illustrates an ultrasound or echo endoscope passing through an anatomical passage and positioned with its distal end close to an anatomical region of interest, according to embodiments.

FIG. 8 is a flow chart depicting a method for delivering treatment using catheter systems as described herein, according to embodiments.

FIG. 9 shows a handle of a needle of a catheter system, according to embodiments.

FIG. 10 is a schematic diagram of an inner assembly of the handle of the needle, according to embodiments.

FIG. 11 is a schematic illustrating a needle in a deployed state, according to embodiments,

FIG. 12 illustrates a method of detecting microswitch engagement, according to embodiments.

### DETAILED DESCRIPTION

The device embodiments of the present disclosure provide device constructions and configurations for the delivery of electroporation or pulsed field ablation therapy for the ablation of soft tissue tumors. In embodiments, the devices are intended for minimally invasive use and in embodiments may be used through the working channel of an endoscopic instrument.

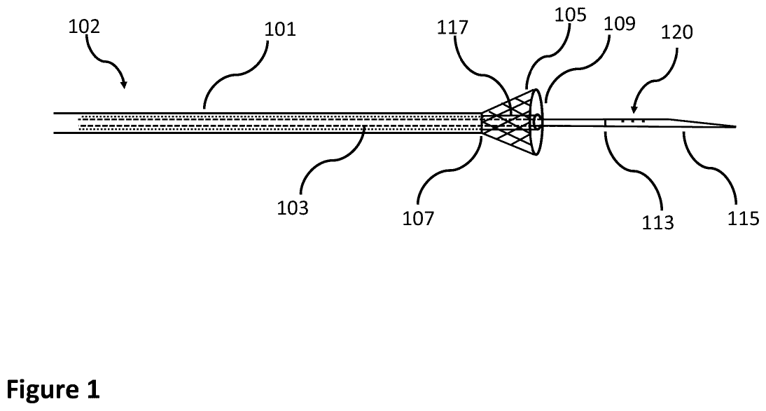

FIG. 1 illustrates a catheter system of the present disclosure, according to embodiments. The catheter system includes a catheter 102 with shaft 101 that has an electrode 105 mounted on its distal portion, where the shaft 101 includes an internal lumen 117. In some embodiments, the internal lumen 117 can be formed or defined by a wall of the shaft 101. In some embodiments, the internal lumen 117 can be formed or defined by an inner tube or shaft of the shaft 101. The wall or other structure that defines the internal lumen 117 can be made of electrically insulating material such as, for example, polymeric electrically insulating materials such as Nylon, PEEK, polyurethane, or similar polymeric materials known in the medical device industry. A needle 103 is disposed within lumen 117 and can comprise a metallic material, such as, for example, stainless steel or Nitinol, and may be insulated over the majority of its proximal portion by a layer of electrically insulating material, such as, for example, PEEK, PFE, parylene, etc. The electrically insulating material covering the needle 103 can terminate at a distal end 113. The distal portion of the needle 115 beyond the distal end 113 of the insulating layer is electrically exposed and has a sharp tip for insertion into tissue and serves as an electrode for delivery of high voltage pulses for pulsed electric field ablation. The electrode 105 is in the form of a self-expanding basket and can comprise a superelastic material such as, for example, Nitinol. In an embodiment, the electrode 105 can comprise a braided construction in the form of a braid formed of Nitinol wires or ribbons, or in alternate embodiments, the electrode 105 can comprise a pattern cut from a tube with portions etched or cut away with a laser to form a tubular structure with struts. The electrode 105 has a proximal end 107 where it attaches to the shaft 101 of the catheter and a distal end 109. The diameter of the distal end 109 of the electrode 105 is larger than the diameter of the proximal end 107. An electrical lead wire (not shown) connects to the proximal end 107 of the electrode 105. The lead wire is insulated with a high dielectric strength material (e.g., a material that can withstand a voltage of at least about 500 Volts across its thickness without dielectric breakdown). The needle 103 is electrically connected near its proximal end or in a catheter handle (not shown) to an electrical lead wire that is similarly insulated with a high dielectric strength material (e.g., a material that can withstand a voltage of at least about 500 Volts across its thickness without dielectric breakdown). The distal portion 115 of the needle and the basket electrode 105 can be configured or used as a bipolar electrode pair for delivery of high voltage pulses for pulsed electric field ablation therapy, wherein a voltage in the range of approximately 500 Volts to approximately 10,000 Volts, including all sub-ranges and values therebetween, can be applied across this bipolar electrode pair in the form of high-voltage electrical pulses. In embodiments, the lead wire coupled to the electrode 105 and the lead wire coupled to the needle 103 can be configured to deliver a voltage waveform, such as the voltage waveforms described for example in International (PCT) Patent Application No. PCT/US2023/025064, titled “Apparatus, Systems and Methods for Soft Tissue Ablation,” filed Jun. 12, 2023, or U.S. patent application Ser. No. 18/976,095, filed Dec. 10, 2024, titled “Apparatus, Systems and Methods for Soft Tissue Ablation,” the disclosure of each of which is incorporated herein by reference.

The wall or other structure that defines the lumen 117 can be configured to extend through a longitudinal length of the catheter 102 and through at least a portion of the basket electrode 105. For example, in some embodiments, the wall or other structure that defines the lumen 117 can extend a full longitudinal length of the basket electrode 105. In such embodiments, the wall or other structure that defines the lumen 117 can be configured to provide insulation and/or prevent contact between portions of the basket electrode 105 and/or the needle 103. Alternatively, the lumen 117 can be configured to terminate at a proximal end of the basket electrode 105.

In some embodiments, the needle 103 can include one or more indentations, grooves, cuts, or other acoustic markings 120 along a portion of its length, which can be visualized via ultrasound imaging (e.g., using an echo-endoscope as described herein). For example, a series of grooves can be disposed on the distal portion 115 of the needle 103 to facilitate visualization of the needle under ultrasound imaging.

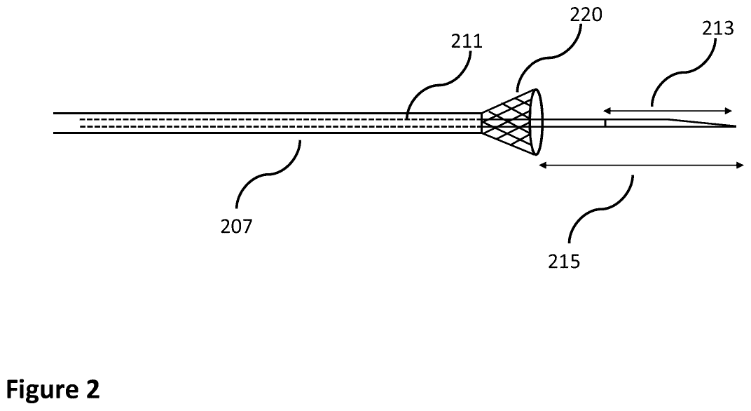

FIG. 2 illustrates a catheter system including a catheter 207, a needle 211, and a basket electrode 220 at the distal end of the catheter shaft, according to embodiments. The catheter system depicted in FIG. 2 can be structurally and/or functionally similar to the catheter system depicted in FIG. 1. For example, the catheter 207 can be structurally and/or functionally similar to the catheter 102, the needle 211 can be structurally and/or functionally similar to needle 103, and he basket electrode 220 can be structurally and/or functionally similar to the electrode 105. In embodiments, the needle 211 extends beyond the distal end of the basket electrode 220 by a length 215 that can range between approximately 5 mm and approximately 80 mm, including all sub-ranges and values therebetween. In some embodiments, the distance that the needle 211 extends beyond the distal end of the basket electrode 220 can depend on the target anatomy, location of the lesion, shape of the lesion, and/or size of the lesion. The proximal portion of the needle 211 is limited in range of motion at the handle of the device (not shown) so that the distally extended portion of the needle 211 can extend out from the catheter by a length 215 that is no more than about 80 mm. The most distal portion of the needle 211, e.g., over a length 213 of the needle 211, is electrically exposed to act as an electrode, while the length of the needle proximal to length 213 is electrically insulated with a high dielectric strength material (e.g., a material that can withstand a voltage of at least 500 Volts across its thickness without dielectric breakdown). In embodiments, the exposed length 213 of the needle can lie in the range between approximately 5 mm and approximately 50 mm, including all sub-ranges and values therebetween. In embodiments, ablation delivery (e.g., pulsed field ablation) can be enabled on a system delivering high voltage pulses to the exposed portion 213 of the needle 211 and the basket electrode 220 when the extended length 215 of the needle is at least about 5 mm. For example, this can be implemented by use of appropriate positioning of electrical contacts in the catheter handle (not shown). For example, electrical contacts that are configured to couple the needle 211 and/or the basket electrode 220 to a generator for delivering high-voltage electrical pulses may couple the needle 211 and/or the basket electrode 220 when each are positioned at the relative distances to one another (e.g., when the needle 211 has been extended the necessary distance from the basket electrode 220). In some embodiments, such electrical contacts may not couple the needle 211 and/or the basket electrode 220 to the generator when the needle 211 has not been extended a predetermined distance from the distal end of the basket electrode 220, e.g., when the needle 211 has not been extended at least about 5 mm beyond the distal end of the basket electrode 220.

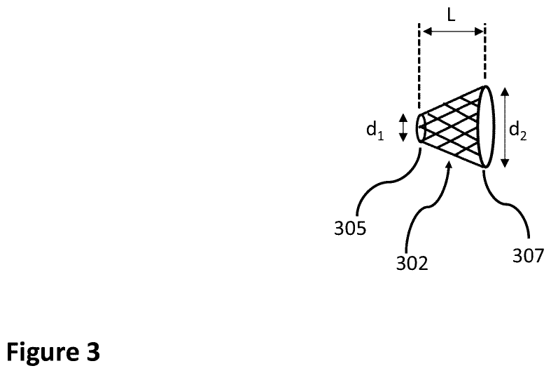

FIG. 3 schematically depicts a basket electrode 302 in an expanded or unstressed state, showing the proximal end 305 of the basket electrode with diameter d1 and distal end 307 of the basket electrode with diameter d2 and with the basket electrode having a length L, according to embodiments. The basket electrode 302 shown in FIG. 3 can be structurally and/or functionally similar to other basket electrodes disclosed herein, including, for example, basket electrode 105 and/or 220. In embodiments, the proximal electrode diameter d1 can lie in the range between approximately 0.5 mm and approximately 10 mm, including all sub-ranges and values therebetween. In embodiments, the distal electrode diameter d2 can lie in the range between approximately 0.8 mm and approximately 20 mm, including all sub-ranges and values therebetween. In embodiments, the ratio of distal diameter to proximal diameter (d2/d1) is at least 1.1. For example, in embodiments, the ratio d2/d1 can lie in the range between approximately 1.1 and approximately 20, including all sub-ranges and values therebetween. In embodiments, the length L of the basket electrode can lie in the range between approximately 2 mm and approximately 20 mm, including all sub-ranges and values therebetween.

As shown in FIG. 3, the basket electrode 302 when expanded or unstressed can be configured to taper across its longitudinal length or have a conical or cone-like shape. For example, the basket electrode 302 can be configured to gradually increase in diameter from its proximal diameter d1 to its distal diameter d2. While the basket electrode 302 is shown to have a tapering structure, it can be appreciated that basket electrodes described herein can be configured to have other shapes and/or configurations. For example, the basket electrode can have a stepped structure, e.g., including a proximal portion that has a first diameter d1 and a distal portion that has a second diameter d2 and one or more stepped increases in diameter between the proximal portion and the distal portion.

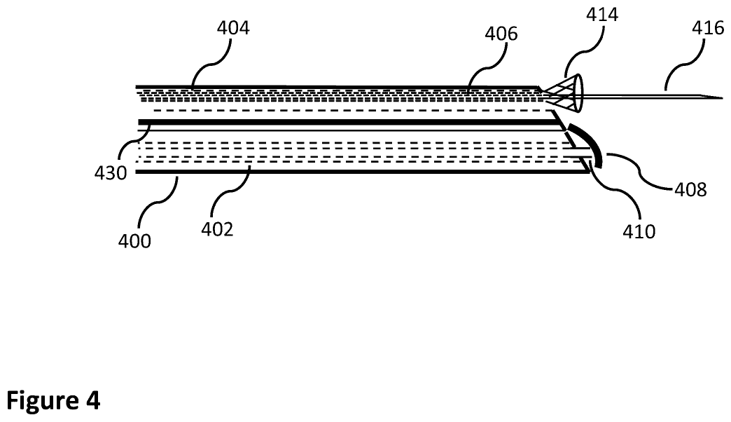

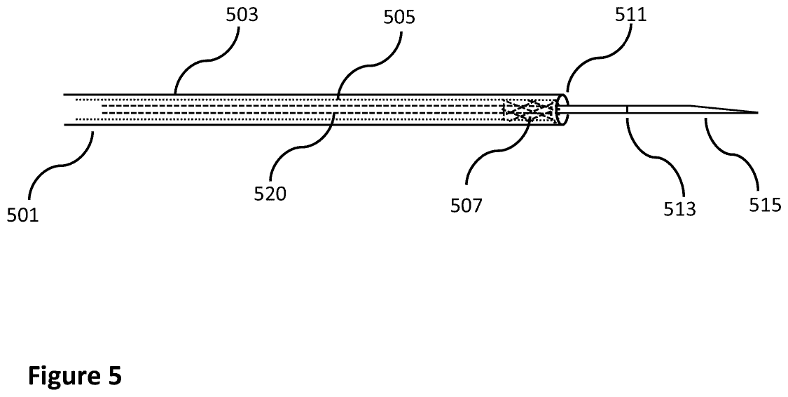

The basket electrode 302 shown in FIG. 3 (and other basket electrodes described herein) can be configured to transition between a first state (e.g., a compressed or undeployed state) and a second state (e.g., an expanded, deployed, uncompressed, or unstressed state). For example, as shown and described with respect to FIGS. 4 and 5, the basket electrode can be compressed within a lumen (e.g., a lumen of a sheath 503, or a working channel 404 of an endoscope 400) for delivery to a target site and then extended distally out of the sheath such that it can expand to its deployed state. In the deployed state, the basket electrode can be configured to deliver pulse field ablation, e.g., with the electrically exposed portion of a needle, as described above. In embodiments, the basket electrode 302 comprises or is formed from a superelastic material, such as, for example, Nitinol, and/or can comprise a braided construction, for example, in the form of a braid formed of Nitinol wires or ribbons. In alternate embodiments, the basket electrode 302 can comprise a pattern cut from a tube (e.g., a Nitinol tube) with portions etched or cut away with a laser to form a tubular, expandable structure with struts.

FIG. 4 schematically illustrates a catheter system or device of the present disclosure passing through a channel of an endoscope, according to embodiments. The endoscope 400 can be, for example, an echo endoscope or echo-endoscope. The figure shows an endoscope 400 with a lumen 402 through which electrical connections 410 are made to an ultrasound transducer 408. Alternatively, or additionally, the endoscope may have a lumen for passage of optical components, or an optical fiber or cable 430. The optical fiber or cable 430 can be configured to couple to an optical imaging element or camera, e.g., to enable capture of optical images of patient anatomy near a distal end of the endoscope 400. The tip of the optical fiber or cable 430 can include imaging components, such as a lens, optical filter, etc. The endoscope also has a working channel 404 through which a catheter system or apparatus 406 of the present disclosure is configured to be passed through to emerge from the distal end of the working channel 404. The catheter system or apparatus 406 can be structurally and/or functionally similar to other catheter systems or devices disclosed herein, including, for example, those depicted in FIGS. 1 and 2. The catheter 406 has a basket electrode 414 (e.g., structurally and/or functionally similar to the basket electrodes 105, 220, or 302) at the distal end of its shaft (e.g., structurally and/or functionally similar to shaft 101). A needle 416 (e.g., structurally and/or functionally similar to needles 103, 211) can be extended out from the catheter 406 to pierce tissue and access a desired target anatomy under ultrasound and/or optical visualization provided by ultrasound transducer 408 and/or optical imaging. In embodiments, the optical imaging modality of the endoscope may generally be used for initial placement of the endoscope, and subsequently the ultrasound imaging modality may be used to visualize deployment and placement of the needle 416. The optical imaging modality can provide visualization of patient anatomy (e.g., a portion of the duodenum), such that the endoscope 400 and/or the catheter system 406 can be navigated to a site adjacent to the tissue or organ to be treated (e.g., the pancreas, liver, kidney, or other adjacent organs). The ultrasound imaging modality can then provide visualization of tissue structures and/or organs that lie underneath or adjacent to the site.

FIG. 5 illustrates an embodiment of the catheter system of the present disclosure, where an outer sheath or shaft is included as part of the catheter system, according to embodiments. The catheter system depicted in FIG. 5 can be structurally and/or functionally similar to other catheter systems or devices disclosed herein, including, for example, those depicted in FIGS. 1, 2, and 4. However, the catheter system depicted in FIG. 5 can include an outer sheath or shaft. FIG. 5 shows a catheter system 501 that comprises an outer sheath 503 through which catheter 505 (e.g., inner shaft) is inserted. The catheter 505 can have a basket electrode 507 (e.g., structurally and/or functionally similar to the basket electrodes 105, 220, 302, 414) disposed at its distal end. In embodiments, the electrode 507 comprises a superelastic material, such as, for example, Nitinol, and can comprise a braided construction, for example, in the form of a braid formed of Nitinol wires or ribbons. In alternate embodiments, the electrode 507 can comprise a pattern cut from a tube with portions etched or cut away with a laser to form a tubular structure with struts. The electrode 507 is flexible and compressible so that it deforms to fit within the sheath 503. To deploy the basket electrode 507, the catheter 505 is pushed at the proximal end so that the distal end of the catheter, and the electrode 507, emerge from the distal end 511 of the sheath 503 (e.g., are extended distal to the distal end of the sheath 503). As the electrode 507 emerges from the sheath 503, it automatically regains its expanded or cone-like shape, e.g., as represented for example by electrode 302 in FIG. 3, electrode 414 in FIG. 4, etc. In some embodiments, the distal end of the basket electrode 507 can be extended between about 3 mm and about 30 mm distally from a distal end of the sheath 503, inclusive of all sub-ranges or values therebetween. A needle 520 (e.g., structurally and/or functionally similar to needles 103, 211, 416) can be configured to pass through an inner lumen of the catheter 505 and can be extended beyond the distal end of the electrode 507, e.g., between approximately 5 mm and approximately 80 mm, including all sub-ranges and values therebetween. As illustrated in FIG. 5, the distal portion 515 of the needle 520 has a sharp tip for puncturing tissue. This distal portion 515 is electrically conducting and comprises a metallic material such as, for example, stainless steel or Nitinol. The proximal portion of the needle is electrically insulated up to the proximal end 513 of the distal portion of the needle by a layer of electrically insulating material such as, for example, PEEK, PFE, parylene, etc. The electrode 507 is connected to an electrical lead wire that is insulated with a high dielectric strength material (e.g., a material that can withstand a voltage of at least about 500 Volts across its thickness without dielectric breakdown). The needle 520 is electrically connected near its proximal end or in a catheter handle (not shown) to an electrical lead wire that is similarly insulated with a high dielectric strength material (e.g., a material that can withstand a voltage of at least about 500 Volts across its thickness without dielectric breakdown).

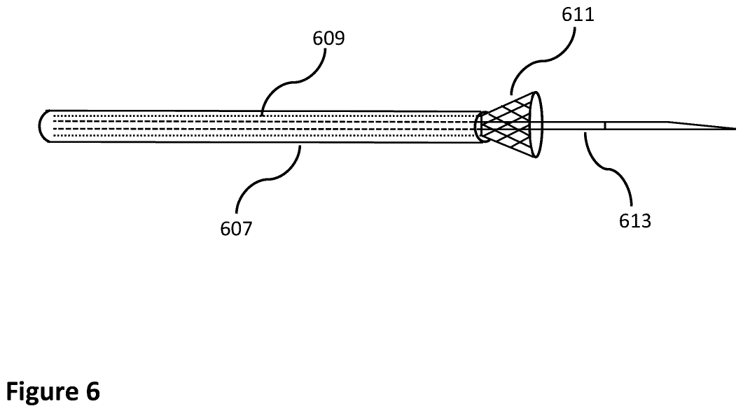

FIG. 6 illustrates an embodiment of the catheter system of the present disclosure, where an outer sheath is included as part of the catheter system, according to embodiments. The catheter system depicted in FIG. 6 can be structurally and/or functionally similar to other catheter systems or devices disclosed herein, including, for example, those depicted in FIGS. 1, 2, 4, and 5. In particular, FIG. 6 shows the basket electrode of FIG. 5 in an expanded state or configuration, e.g., after the basket electrode has been extended beyond a distal end of the sheath.

FIG. 6 depicts a catheter system with an outer sheath 607 (e.g., structurally and/or functionally similar to the sheath 503) through which a catheter 609 can pass. The catheter 609 has a deployed basket electrode 611 (e.g., structurally and/or functionally similar to the basket electrodes 105, 220, 302, 414, 507) disposed at its distal end. When the catheter is pushed forward such that the basket electrode 611 extends beyond the distal end of the sheath 607, the electrode 611 is free to expand and assumes its original or unstressed partial cone-like shape, as indicated by 611, wherein the distal end of the basket electrode has a larger diameter than its proximal end. Such can be similar to that described with respect to basket electrode 302 in FIG. 3. Needle 613 (e.g., structurally and/or functionally similar to needles 103, 211, 416, 520) can be extended through the catheter and the basket electrode 611 to emerge beyond the distal end of the basket electrode 611, as illustrated in FIG. 6. In embodiments, the catheter system, including the outer sheath 607, can be passed through the working channel of an endoscope as a single unit. While the needle 613 is shown passing through the electrode 611, in alternative embodiments, the needle 613 can pass through an inner or internal lumen of the catheter 609, and the inner lumen can extend to or nearly to the distal section of the basket electrode 611. For example, this was depicted and described with respect to lumen 117 in FIG. 1.

In use, once the sheath 607 is positioned at or close to the distal end of the endoscope channel, the catheter (e.g., catheter 505, 609, or any other catheter described herein) can be moved forward to deploy the basket electrode (e.g., 105, 220, 302, 414, 507, 611). The endoscope can be suitably articulated to position the basket to face a desired orientation. Once the basket electrode is so deployed (e.g., extended to its deployed state), the needle (e.g., 103, 211, 416, 520, 613) can be extended to a desired anatomical target location, e.g., with assistance as needed from visualization with ultrasound imaging from the ultrasound transducer.

In some embodiments, an annular space defined between an inner surface of the sheath 607 and an outer surface of the catheter 609 (or shaft of the catheter) can be used to deliver a fluid or other agents to a region or zone around the expandable electrode 611. For example, the annular space can be configured to deliver a conductive fluid such as saline to the region around the expandable electrode 611, e.g., to envelop or substantially envelop the expandable electrode 611 in the fluid. In use, when the needle tip is deployed in a tissue structure or organ and the expandable electrode 611 is disposed outside of but adjacent to the tissue structure, the fluid can ensure that a conductive path is established between the expandable electrode 611 and the needle tip. Such can ensure that pulsed field ablation therapy can be effectively delivered to the target region of the tissue structure.

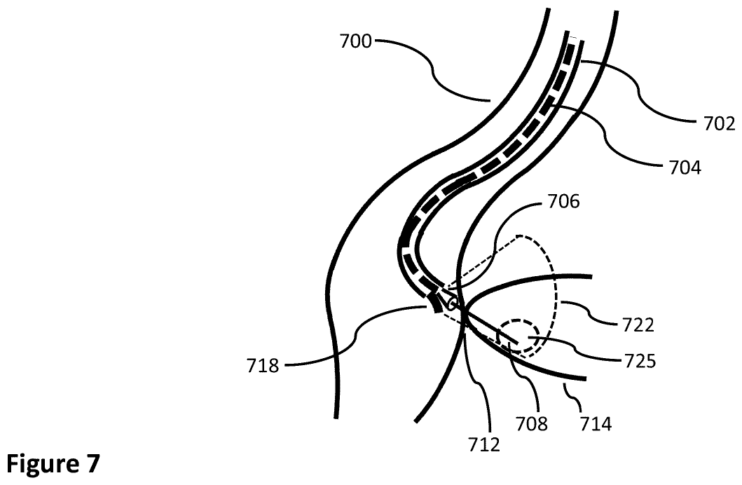

FIG. 7 schematically illustrates an ultrasound or echo endoscope passing through an anatomical passage and positioned with its distal end close to an anatomical region of interest, according to embodiments. Specifically, the figure shows an endoscope 702 (e.g., structurally and/or functionally similar to the endoscope 400) passing through an anatomical passage 700. A catheter system 704 of the present disclosure (e.g., structurally and/or functionally similar to any of the catheter systems or devices described herein) passes through a working channel of the endoscope 702, and a distal portion of a catheter of the catheter system 704 extends from the distal end of the endoscope 702. The extended portion of the catheter system 704 can include a basket electrode 706 (e.g., structurally and/or functionally similar to basket electrodes 105, 220, 302, 414, 507, 611). The endoscope 702 also has an ultrasound transducer 718 (e.g., structurally and/or functionally similar to ultrasound transducer 408) disposed at its distal end. The endoscope 702 is placed in a region of interest with a surface boundary 712. For example, the endoscope can be placed in a stomach or duodenum. A needle 708 passes through the catheter and is extended to puncture the surface boundary 712 and enter into an adjacent organ 714 (for example, the pancreas, liver, lung, or other soft tissue organs). The extension of the needle 708 can be performed under visualization, e.g., from the ultrasound transducer which can generate an image region 722 that is displayed on a monitor or screen. The needle 708 can be visualized on such an image and the needle 708 can be directed towards an anatomical region of interest, such as the region 725 in organ 714 (for example, this can be a tumor in the pancreas, liver, lung, etc. where it is desired to deliver ablation therapy such as pulsed field ablation therapy). Once the needle 708 is suitably positioned, pulsed electric field ablation therapy can be delivered in the form of high voltage pulses delivered to the bipolar electrode pair comprising needle electrode 708 and basket electrode 706. Such high voltage pulses with a voltage amplitude in the range between approximately 500 V and approximately 10,000 V, including all sub-ranges and values therebetween, are described for example in International (PCT) Patent Application No. PCT/US2023/025064 and U.S. patent application Ser. No. 18/976,095, incorporated above by reference.

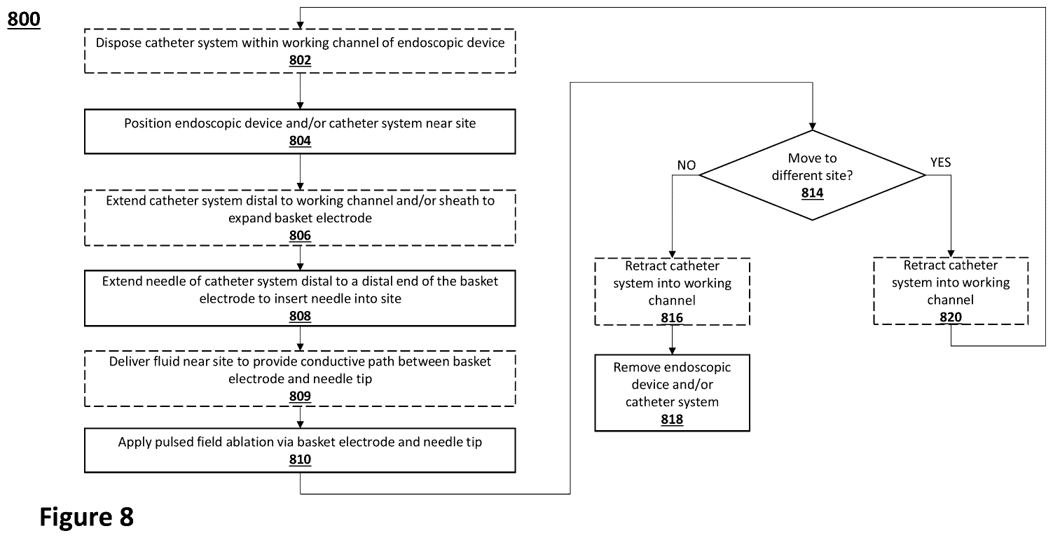

FIG. 8 depicts a method 800 of using the endoscopic devices and catheter systems described herein (e.g., any of the endoscopic devices depicted in FIGS. 4 and 7 and/or catheter systems and/or components depicted in FIGS. 1-7), according to embodiments. At 802, a catheter system can optionally be disposed within a working channel of an endoscopic device. Alternatively, a catheter system may be positioned outside of an endoscopic device, e.g., alongside or adjacent to an endoscopic device. In some embodiments, a catheter system may be used without an endoscopic device. At 804, the endoscopic device and/or catheter system can be positioned near a tissue site, e.g., by navigating the endoscopic device and/or catheter system through a body lumen or cavity. The tissue site can include a tissue mass, nodule, or tumor, or other element that requires treatment, e.g., in the pancreas, liver, kidney, lung, etc. In some embodiments, the catheter system can include an expandable electrode (e.g., any of the basket electrodes described herein). The expandable electrode may be held in an undeployed or constrained state within the working channel of the endoscopic device and/or within an outer sheath, while the endoscopic device is being navigated to the tissue site. In some embodiments, the endoscopic device can be configured to provide image guidance (e.g., via any of the ultrasound transducers and/or optical imaging components described herein) for positioning the catheter system at the target site. For example, the endoscopic device can include an optical fiber (e.g., optical fiber 430), which can provide a view of patient anatomy (e.g., a portion of the duodenum) as the device is navigated therein to a particular site.

At 806, if the catheter system is disposed within the working channel of the endoscopic device, the catheter system can be extended out of the working channel. Alternatively, or additionally, a catheter of the catheter system can be extended distally relative to an outer sheath of the catheter system to deploy or expand the expandable electrode. The catheter system, as described above, can include a needle. At 808, the needle of the catheter system can be extended out from a distal end of the expandable electrode such that a distal penetrating tip of the needle can be inserted into the tissue site. In some embodiments, the needle can include one or more markings (e.g., markings 120), which can enable visualization of the needle via ultrasound when the needle is being extended. The ultrasound imaging can therefore be used to ensure proper deployment and positioning of the needle within target tissue (e.g., an organ adjacent to the duodenum).

Optionally, at 809, a fluid such as a conductive fluid may be delivered to a region surrounding the expandable electrode. In some embodiments, a sheath (e.g., sheath 607) and a catheter (e.g., catheter 609) can define an annular space that is configured to deliver fluids or other agents to the region surrounding the expandable electrode. In some embodiments, the working channel of the endoscopic device (or another channel of the endoscopic device) can be configured to deliver fluids or other agents to the region surrounding the expandable electrode. In some embodiments, one or more separate devices (e.g., another catheter) can be configured to deliver fluids or other agents to the region surrounding the expandable electrode. The fluid, once delivered, can be configured to surround the expandable electrode to provide or establish at least a portion of a conductive path between the expandable electrode and the tip of the needle. For example, the fluid can ensure that any hollow empty space inside the body lumen within which the expandable electrode is located (e.g., a portion of the duodenum) is filled with the fluid and therefore able to conduct a current. The needle can be inserted into adjacent or neighboring tissue structures or organs, which can include tissue structures and fluids that are conductive. Together, the fluid around the expandable electrode and the conductive tissue or fluids in the adjacent tissue structures can provide or establish a conductive path between the expandable electrode and the tip of the needle, thereby enabling effective and/or efficient delivery of pulsed field ablation.

At 810, the catheter system, via the expandable electrode and the distal tip of the needle, can apply pulsed field ablation or electroporation to a zone of tissue at the tissue site. For example, a generator can be configured to deliver voltage waveforms to the catheter device, to energize one or more electrodes of the catheter device to deliver electroporation. In some embodiments, the generator can be configured to cause the one or more electrodes to generate an electric field having sufficient strength to cause irreversible electroporation or pulsed field ablation. In some embodiments, the generator can be configured to cause the electrodes to reversibly electroporate tissue.

In some embodiments, the catheter system can repeat the application of electroporation one or more times. For example, electroporation can be repeated at a given site as needed for an enhanced ablation effect. In some embodiments, the catheter system can be moved to one or more additional sites (814: YES) to deliver electroporation to those sites. For each new site, the catheter system may be withdrawn into the working channel of the endoscopic device and/or the needle may be withdrawn into the catheter shaft, at 820, and the endoscopic device and/or catheter system can be moved to the new tissue site, at 804. In some cases, the needle can be withdrawn in steps corresponding to distinct needle extensions, with ablation being delivered at each step or extension. The process of inserting the needle into the tissue site and delivering electroporation can then repeat, at 806-814. In some embodiments, the catheter device can be moved to between 1 and about 15 different sites. When the procedure is completed, i.e., no further tissue sites need to be ablated (814: NO), then if the catheter system was delivered via a working channel of an endoscopic device, the catheter system can be optionally retracted into the working channel, at 816, and the entire assembly including the endoscopic device and the catheter system can be removed from the patient, at 818. In embodiments where the catheter system was used independently of an endoscopic device, the needle can be retracted into the catheter shaft and/or the expandable electrode can be retracted into the outer sheath, and the catheter system can be removed form the patient, at 818.

As can be appreciated, each of 802-818 can be performed with image guidance. For example, the endoscopic device can be configured to capture images of the tissue site or catheter system and/or components thereof to ensure proper positioning of the catheter system and/or components thereof prior to delivering electroporation. In some embodiments, the endoscopic device can be an echo endoscope and can include an ultrasound transducer to provide ultrasound imaging, e.g., to confirm insertion of the needle of the catheter system into a tissue site. In some embodiments, the endoscopic device may include an imaging channel where an imaging device can be extended therethrough to capture images of the tissue site and/or catheter system. In some embodiments, a separate imaging device or mechanism can be used to view and confirm the position of the catheter system relative to the tissue and the delivery of electroporation. In some embodiments, the imaging device (as extended through the endoscopic device or separately disposed near the tissue site) can be configured to be deflected, e.g., to capture different views around the tissue site. The imaging modality can comprise direct optical imaging, ultrasound imaging, or other imaging modalities as typically employed in the art.

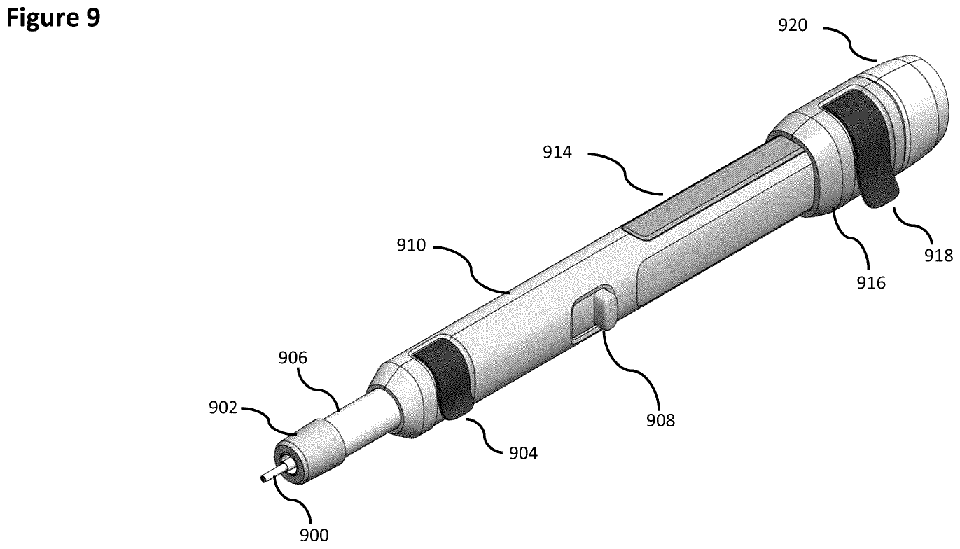

FIG. 9 shows a handle 910 coupled to a proximal end of an outer catheter shaft 900, according to an embodiment. The handle 910 can be used with any of the catheter systems described herein, including for example, those depicted in FIGS. 1-7 and 11.

The handle 910 can include one or more deployment mechanisms to actuate a needle slidably disposed in an outer catheter shaft 900 of a catheter system as described herein. As illustrated in FIG. 9, the handle 910 includes a scope shaft portion 906 (e.g., a shaft) configured to be disposed around a proximal portion of the outer catheter shaft 900. The scope shaft portion 906 can include a scope tip portion 902 (e.g., a tip) at a distal end thereof. The scope tip portion 902 can be configured to engage with an endoscopic device (not shown), such as, for example, an instrument port that leads into a working channel of an endoscopic device. The endoscopic device can be structurally and/or functionally similar to endoscopes 400, 702. The scope tip portion 902 may include a first locking mechanism or locking device (not shown). In some embodiments, the first locking mechanism can include, for example, a threaded or screw portion for engaging with a Luer lock on the endoscope. The scope tip portion 902 can be configured to couple to the endoscopic device such that the outer catheter shaft 900 is disposed within the working channel of the endoscopic device. For example, the scope tip portion 902 can be coupled to an instrument port of the endoscopic device such that the outer catheter shaft 900 (which extends distally from the scope tip portion 902) is disposed within the working channel. In some embodiments, the scope shaft portion 906 can be configured to move (e.g., slide, extend, etc.) longitudinally relative to the housing of the handle 910. The scope shaft portion 906 can be extended over the outer catheter shaft 900 at a range of lengths by sliding the scope shaft portion 906. Therefore, the scope tip portion 902 can be disposed around different positions along a length of the outer catheter shaft 900 to accommodate endoscopes with different working channel lengths so that the appropriate length of the outer catheter shaft 900 can be fully engaged or enclosed in the working channel of a given endoscope. For example, to accommodate an endoscope with a shorter working channel length, the scope shaft portion 902 can be extended distally over the outer catheter shaft 900 such that a length of a portion of the outer catheter shaft 900 disposed distal to the scope shaft portion 902 is set appropriately based on a length of the working channel of the endoscope. In some instances, it may be desirable to have the length of the outer catheter shaft 900 that extends distally from the scope shaft portion 902 be equal to or substantially equal to the length of the working channel, such that the distal end of the outer catheter shaft 900 terminates at or near the distal end of the working channel. In some instances, it may be desirable to have the length of the outer catheter shaft 900 that extends distally from the scope shaft portion 902 be greater than the length of the working channel, such that the distal end of the outer catheter shaft 900 extends distally by a predetermined distance from the distal end of the working channel. The endoscope can be locked in place relative to the outer catheter shaft 900 by engaging the Luer lock of scope tip portion 902 with a portion of the endoscopic device (e.g., an instrument port of the endoscopic device). In embodiments, the scope shaft portion 906 can include a ruler or length markings to indicate a length of the scope shaft portion 906 that is extending from the housing of the handle 910.

The scope shaft portion 906 can be locked to the handle 910 with a second locking mechanism or locking device 904. The second locking mechanism 904 in the unlocked configuration can allow the scope shaft portion 906 to slide longitudinally relative to the housing of the handle 910 such that the scope tip portion 902 can be disposed around the outer catheter shaft 900 at a desired position corresponding to the length of the working channel of the endoscope. In some embodiments, the second locking mechanism 904 in the locked configuration prevents the scope shaft portion 906 from moving (e.g., sliding) relative to the housing of the handle, and therefore locks the scope tip portion 902 at the desired position relative to the outer catheter shaft 900. In some embodiments, the second locking mechanism 904 can include a cam lock mechanism with a hinge that can be opened to release and slide the scope shaft portion 906 or closed and snapped shut to lock scope shaft portion 906 in place. In some embodiments, the second locking mechanism 904 can include a friction lock with an actuator such as a button to release friction and enable sliding. In some embodiments, the second locking mechanism 904 can include a set screw that is unscrewed to release (e.g., and allow movement of the scope shaft portion 906) or screwed tight to lock the scope shaft portion 906 in place. In can be appreciated that other configurations of locking mechanisms can also be used herein, including locking mechanisms that allow for movement of the scope shaft portion 906 to preset positions (e.g., positions corresponding to different length working channels or different endoscopic devices associated therewith), or locking mechanisms that allow for continuous movement of the scope shaft portion 906 such that the scope shaft portion 906 can be set to any position along a range of positions relative to the catheter shaft 900. In some embodiments, an outer surface of the scope shaft portion 906 may include one or more markings to indicate positions to lock the scope shaft portion 906, e.g., for having the length of the catheter shaft 900 that extends distally from the scope shaft portion 906 be set at a predetermined length for endoscopic devices having a known working channel length.

In some embodiments, the handle 910 can include a first actuator, such as, for example, a basket extension mechanism 908, which is coupled to an inner catheter shaft of the catheter system. The inner catheter shaft can include an electrode (e.g., a basket electrode as described herein). The inner shaft and electrode can be structurally and/or functionally similar to any of the shafts or electrodes described herein such as shaft 101 and electrode 105. In some embodiments, the basket extension mechanism 908 can be configured to extend the inner catheter shaft with the electrode attached at the distal end of the inner catheter shaft distal to the outer catheter shaft 900 and/or endoscope to deploy the electrode. For example, the basket extension mechanism 908 can advance the electrode distal to the outer catheter shaft 900 and/or endoscope such that the electrode transitions from a first configuration in which the electrode is constrained in the a lumen of the outer catheter shaft 900 and/or a working channel of the endoscope and unexpanded to a second configuration in which the electrode is unconstrained and expands (e.g., into a basket shape). In embodiments, the basket extension mechanism 908 can be implemented as a slider, a knob, or any suitable actuator configured to cause linear movement of the inner catheter shaft. In some embodiments, the basket extension mechanism 908 can include a slider 908 as shown in FIG. 9. The basket extension mechanism 908 can also be configured to retract the inner catheter shaft relative to the outer catheter shaft such that the electrode attached at the distal end of the inner catheter shaft is retracted back into the outer catheter shaft. When the electrode is a basket electrode as described herein, the electrode can collapse back into the outer catheter shaft when retracted. Alternatively, in some embodiments, an actuator can be coupled to the outer catheter shaft and be configured to retract and/or extend the outer catheter shaft relative to the inner catheter shaft to deploy the electrode disposed at the distal end of the outer catheter shaft (e.g., to allow the basket electrode to expand or collapse).

In some embodiments, the handle 910 includes a second actuator, such as, for example, a needle grip 916, that is configured to deploy the needle of the catheter system. In some embodiments, the needle grip 916 can be disposed around a proximal portion of the housing of the handle 910. The needle grip 916 can be coupled to a proximal end of the needle and can be configured to move (e.g., slide) along the handle 910 to move the needle longitudinally. For example, the needle grip 916 can be configured to slide along a track 914 disposed on the housing of the handle 910. In some embodiments, the track 914 can include ruler or length markings to indicate a length of the needle that is deployed. The length of the needle that is deployed refers to a length of the needle that is disposed distal to the distal end of the deployed basket electrode at the distal end of the catheter shaft that is at the distal end of the endoscope, for example the deployed needle length 215 indicated in FIG. 2. In embodiments the ruler markings can include a fixed, predetermined offset relative to the deployed length of the needle. The needle grip 916 includes a third locking mechanism or locking device 918 configured to transition between an unlocked configuration, in which the third locking mechanism 918 releases the needle grip 916 so that the needle grip 916 can slide over the needle portion, and a locked configuration, in which the third locking mechanism 918 to locks the needle grip 916 in place. In some embodiments, the locking mechanism 918 can include a cam lock mechanism with a hinge that can be opened to release and slide the needle grip 916 or closed and snapped shut to lock the needle grip 916 in place (e.g., similar to the cam lock mechanism described above with respect to the locking mechanism 904 of the scope shaft portion 906). In some embodiments, the third locking mechanism 918 can be a friction lock with an actuator such as a button to release friction and enable sliding. In some embodiments, the third locking mechanism 918 can be a set screw that is unscrewed to release or screwed tight to lock the needle grip 916. In some embodiments, the needle grip 916 includes an endcap 920 configured to be attached to an electrical cable and/or a fluid port. The endcap 920 can be disposed at the proximal end of the needle grip 916. In embodiments, the endcap can include an electrical port or coupler for coupling to an electrical cable (e.g., a plug). In some embodiments, the endcap can include a port for coupling to a fluid line.

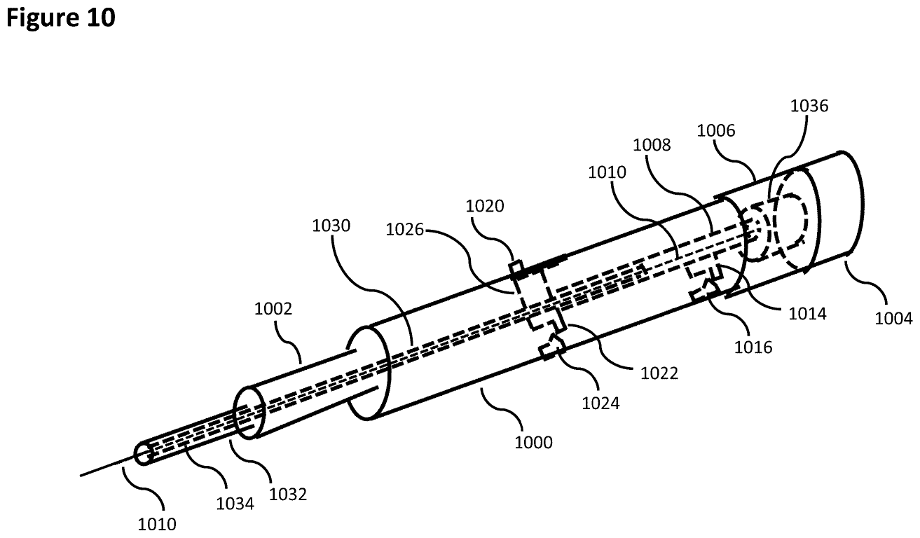

FIG. 10 is a schematic diagram of an inner assembly of a handle of a catheter system, according to embodiments. FIG. 10 shows a catheter handle 1000 with a scope shaft portion (e.g., shaft) 1002 configured to move (e.g., longitudinally) over a portion of the outer catheter shaft 1032. The handle can include a first actuator (e.g., basket extension slider) 1020 and a second actuator (e.g., needle grip portion) 1006. In some embodiments, the needle grip portion 1006 is configured to be manipulated to deploy a needle 1036. The needle 1036 can include an electrode on a distal portion thereof. For example, a distal tip of the needle 1036 can include an exposed conductive material. The needle grip portion 1006 can include endcap 1004 (e.g., similar to endcap 904). Inner catheter shaft (depicted with proximal portion 1030 and distal portion 1034), passes through the outer catheter shaft 1032 and can include an electrode (e.g., a basket electrode) at its distal end (not shown). A proximal portion 1030 of the inner catheter shaft can be disposed in the handle and coupled to a basket carrier 1026, which can include a contoured surface 1022. In some embodiments, the basket carrier 1026 can be configured to move through the housing with the inner catheter shaft as the first actuator actuates the inner catheter shaft. In some embodiments, when the first actuator (e.g., the basket extension slider) 1020 is at a maximum distal position such that the basket electrode is deployed, the contoured surface 1022 of the carrier 1026 engages a first switch, such as, for example, a microswitch 1024, that is mounted inside the handle 1000, thereby transitioning the first microswitch 1024 to an engaged configuration or position. In some embodiments, the first microswitch 1024 can be operatively coupled to a generator. In some embodiments, the generator can be configured to detect when the first microswitch 1024 is in the engaged position, which confirms complete or full basket electrode deployment.

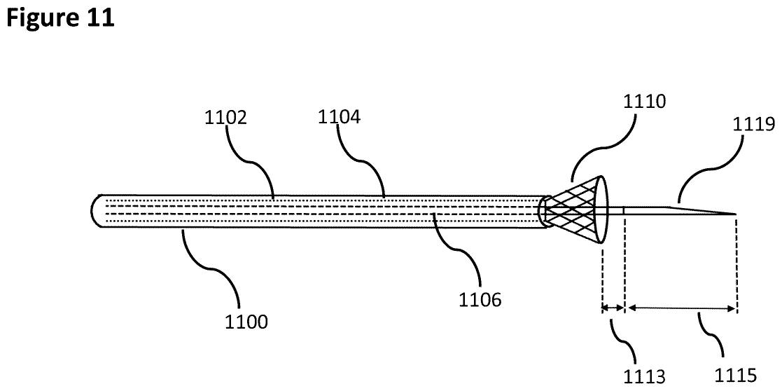

In some embodiments, the needle 1010 passes through the inner catheter shaft 1034 and is coupled to an interior portion 1036 of the endcap 1004 and needle grip 1006. A tubular section 1008 telescopes over the inner catheter shaft 1030 when the needle 1010 is deployed and a needle carrier portion 1014 is attached to tubular section 1008. Therefore, the needle carrier portion 1014 is configured to move through the housing when the needle 1010 is moved. In some embodiments, a second switch, such as, for example, microswitch 1016, is mounted inside the handle 1000 and is configured to be engaged by the needle carrier portion 1014 when the needle carrier portion 1014 is in a position (e.g., a distal position) corresponding to a minimum needle deployment (e.g., as described with reference to FIG. 11 below). As used herein, the minimum needle deployment refers to a position of the needle 1010 when the distal end of the needle 1010 (e.g., an exposed portion) is disposed at least a predetermined distance (e.g., the minimum separation 1113 shown in FIG. 11 below) beyond a distal end of the inner catheter shaft 1034 (and therefore a distal end of the electrode basket). In some embodiments, the engaged configuration or position of the second microswitch 1016 confirms minimum needle deployment (e.g., when an exposed portion of the needle 1010 is at least the predetermined distance from the basket electrode). The engaged position of the second microswitch 1016 can be detected by the generator.

In some embodiments, when the electrode and needle are suitably deployed (e.g., when the electrode and the needle have been extended their predetermined distances from the outer catheter shaft, respectively), the generator can be configured to deliver high voltage pulsed field ablation energy via the electrode and/or the needle. The engagement of both microswitches 1016 and 1024 can confirm that the catheter system (e.g., both the electrode of the inner catheter shaft 1034 and the needle 1010) is suitably deployed to enable ablation delivery. Partial deployment of the basket electrode can result in inadequate energy delivery. In another scenario, if the exposed portion of the distal tip of the needle electrode is not adequately spaced from the basket electrode, arcing can occur. It is desirable to avoid both of these situations. In some embodiments, the first microswitch 1024 being in the engaged configuration indicates that the basket is fully deployed (e.g., has transitioned from an unexpanded configuration to an expanded configuration), and the second microswitch 1016 being in the engaged configuration indicates that the distal tip of the needle 1010 and a distal end of the basket electrode have at least the minimum predetermined distance therebetween (e.g., as described with respect to FIG. 11) such that energy can be properly delivered. Therefore, detection of suitable deployment of both the basket electrode and the needle electrode as described in the present disclosure provides a safety check to ensure adequate and proper ablation is delivered.

FIG. 11 illustrates a catheter system including an outer catheter shaft 1100, inner catheter shaft 1102 and needle 1106. The catheter system depicted in FIG. 11 can be structurally and/or functionally similar to other catheter systems described herein, including, for example, those described with reference to FIGS. 1-7. Annular space 1104 between the inner catheter shaft 1102 and outer catheter shafts 1100 can be configured to convey fluid for fluid irrigation. In some embodiments, basket electrode 1110 is mounted at the distal end of the inner shaft 1102. The catheter system 1110 can further include an electrical lead wire (not shown) with high dielectric strength insulation that can run in the annular space 1104 from the proximal handle to the distal portion of the inner catheter shaft 1102 and can be electrically connected to a proximal portion of the basket electrode 1110 to a generator. In embodiments, the lead wire insulation can withstand a voltage potential difference of at least about 300 V across its thickness without undergoing dielectric breakdown, inclusive of all subranges and values therebetween. The needle 1106 is shown deployed such that the electrically exposed distal electrode portion 1119 of the needle with length indicated by 1115 is extended beyond the distal end of the basket by at least a minimum separation (or distance) 1113. In embodiments the minimum separation of the exposed electrode portion 1119 of the needle from the distal end of the basket 1110 can be between about 0.5 mm and about 10 mm, inclusive of all values and subranges therebetween. When the catheter system is used with the handle as depicted and described herein (e.g., with reference to FIGS. 9 and 10), the microswitch 1016 in FIG. 10 can be suitably located relative to the needle carrier 1014 such that this minimum separation can be detected by engagement of the microswitch 1016 by the needle carrier 1014.

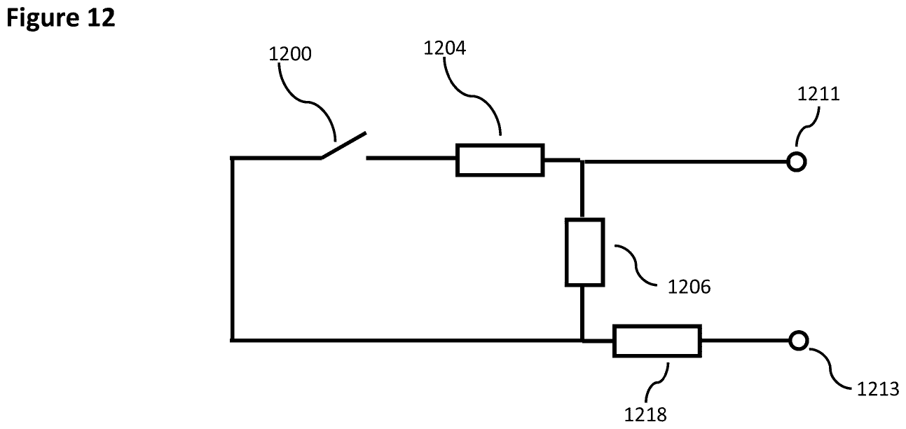

FIG. 12 illustrates a method of detecting microswitch engagement, according to embodiments. In some embodiments, the switch 1200 is connected to voltage divider resistors 1204 and 1206 and terminals 1211 and 1213 as shown in FIG. 12. When a DC voltage is applied across the terminals 1211 and 1213, the resulting current across the terminals 1211, 1213 depends on the switch position, and a measurement of this current or an equivalent voltage measurement across a voltage sense resistor 1218 can indicate the microswitch is engaged (e.g., in the engaged configuration). It should be apparent that when either the basket or the needle is withdrawn to an inadequately deployed configuration, the respective microswitch becomes disengaged and this situation is detected by the circuitry.

This circuitry or alternate embodiments may be included on a chip incorporated into the handle (e.g., the handles described in FIGS. 9-11), or alternately it may be incorporated on a pulse generator that the catheter system connects to for delivery of high voltage pulses for pulsed field ablation. In embodiments, when the user engages an “Ablate” button on the generator user interface or otherwise provides a user input for delivery of ablation to a distal end of the catheter system (e.g., the basket electrode and needle), the pulse generator console first determines if both microswitches 1024 and 1016 in FIG. 10 are engaged, indicating correct deployment. If correct deployment is confirmed, the generator proceeds to deliver ablation. If incorrect deployment is detected (for example, one or both of the microswitches are not engaged), a warning message is displayed on the user interface (or other output device) informing the user to check and correct device deployment and ablation is not delivered. In this manner, the console can implement safe and effective ablation delivery. Therefore, the generator may be prevented from delivering a waveform, and therefore the distal end of the catheter system is prevented from delivery of ablation, until both microswitches 1024, 1016 shown in FIG. 10 are engaged.

While specific examples have been provided in the figure for example and illustrative purposes, it should be clear that variants such as different numbers of lumens, different numbers of electrodes, different electrodes, catheter and wire diameters, different electrode and shaft materials, and the like can be constructed without limitation and based on the teachings herein.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

As used herein, the terms “about” and/or “approximately” when used in conjunction with numerical values and/or ranges generally refer to those numerical values and/or ranges near to a recited numerical value and/or range. In some instances, the terms “about” and “approximately” may mean within ±10% of the recited value. For example, in some instances, “about 100 [units]” may mean within ±10% of 100 (e.g., from 90 to 110). The terms “about” and “approximately” may be used interchangeably.

1. An apparatus, comprising:
an outer shaft defining a first lumen;
an inner shaft disposed within the first lumen and extendable relative to the outer shaft, the inner shaft defining a second lumen;
an expandable electrode disposed on a distal end of the inner shaft, the expandable electrode configured to have a conical shape that increases in diameter from a proximal end to a distal end of the expandable electrode, the expandable electrode is configured to automatically transition from a first unexpanded configuration to a second expanded configuration having the conical shape when the expandable electrode is disposed distal to the outer shaft; and
a needle disposed within the second lumen and extendable relative to the inner shaft, the needle including a distal tip that is electrically exposed and a section proximal of the distal tip that is electrically insulated, the distal tip of the needle being insertable into a tissue site, the expandable electrode and the distal tip of the needle, when (1) the expandable electrode is disposed distal to the outer shaft and (2) the needle is extended relative to the inner shaft such that the distal tip of the needle is disposed distally of the distal end of the inner shaft, being configured to apply pulsed field ablation to a zone of tissue at the tissue site.

2. The apparatus of claim 1, further comprising:
a first lead wire configured to couple the expandable electrode to a generator; and
a second lead wire configured to couple the needle to a generator, such that the generator can deliver a pulse waveform to the expandable electrode and the needle to cause the expandable electrode and the needle to function as a bipolar electrode pair.

3. The apparatus of claim 2, wherein the first lead wire and the second lead wire are electrically insulated lead wires having an insulating layer that is configured to withstand at least about 300 Volts across a thickness of the insulating layer without dielectric breakdown

4. The apparatus of claim 1, wherein the expandable electrode is formed of a superelastic material.

5. The apparatus of claim 1, wherein the second lumen extends along a portion of the expandable electrode. The apparatus of claim 1, wherein the expandable electrode has a first diameter at the proximal end of the expandable electrode and a second diameter at the distal end of the expandable electrode, and a ratio of the second diameter to the first diameter is at least about 1.1.

7. The apparatus of claim 1, wherein the needle is configured to be extended relative to the inner shaft such that the distal tip of the needle is disposed up to about 80 mm from the distal end of the expandable electrode.

8. The apparatus of claim 1, wherein the expandable electrode, when the distal tip of the needle is inserted into the tissue site, is configured to be spaced from the tissue site.

9. The apparatus of claim 1, wherein the outer shaft and the inner shaft define an annular space therebetween that is configured to deliver a conductive fluid to a region surrounding the expandable electrode to establish at least a portion of a conductive path between the expandable electrode and the distal tip of the needle.

10. The apparatus of claim 1, further comprising:
a first switch operatively coupled to the inner shaft and configured to be transitioned from a disengaged configuration to an engaged configuration when the expandable electrode is disposed distal to the outer shaft to indicate the expandable electrode has transitioned to the expanded configuration; and
a second switch operatively coupled to the needle and configured to be transitioned from a disengaged configuration to an engaged configuration when the distal tip of the needle is disposed at least a minimum distance from a distal end of the expandable electrode to indicate the pulsed field ablation can be applied.

11. An apparatus, comprising:
an outer shaft defining a first lumen;
an inner shaft disposed within the first lumen and extendable relative to the outer shaft, the inner shaft defining a second lumen;
an expandable electrode disposed on a distal end of the inner shaft; and
a needle disposed within the second lumen and extendable relative to the inner shaft, the needle including a distal tip that is electrically exposed and a section proximal of the distal tip that is electrically insulated, the distal tip of the needle configured to be advanced distal to the expandable electrode and inserted into a tissue site,
the outer shaft and the inner shaft defining an annular space therebetween that is configured to deliver a conductive fluid to a region surrounding the expandable electrode,
the expandable electrode and the distal tip of the needle, when the needle is inserted into the tissue site and the conductive fluid surrounds the expandable electrode, being configured to apply pulsed field ablation via a conductive path established by the conductive fluid to a zone of tissue at the tissue site.

12. The apparatus of claim 11, wherein the expandable electrode in an expanded configuration has a conical shape in which a diameter of the expandable electrode increases distally.

13. The apparatus of claim 11, wherein the expandable electrode, when the distal tip of the needle is inserted into the tissue site, is configured to be spaced from the tissue site.

14. The apparatus of claim 11, wherein the expandable electrode and the distal tip of the needle are configured to be electrically polarized with opposite polarities to apply the pulsed field ablation to the zone of tissue.

15. The apparatus of claim 11, wherein the expandable electrode is configured to automatically transition from a first unexpanded configuration to a second expanded configuration having a conical shape when the inner shaft is extended relative to the outer shaft such that the expandable electrode is disposed distal to a distal end of the outer shaft

16. The apparatus of claim 11, further comprising:
a first lead wire configured to couple the expandable electrode to a generator; and
a second lead wire configured to couple the needle to a generator, such that the generator can deliver a pulse waveform to the expandable electrode and the needle to cause the expandable electrode and the needle to function as a bipolar electrode pair.

17. The apparatus of claim 11, wherein the outer shaft is coupled to an ultrasound transducer, the ultrasound transducer configured to provide ultrasound imaging to facilitate visualization of one or more portions of at least one of the apparatus or patient anatomy.

18. The apparatus of claim 17, wherein the ultrasound transducer configured to provide ultrasound imaging to facilitate visualization of insertion of the distal tip of the needle into the tissue site.

19. The apparatus of claim 11, further comprising:
a first switch operatively coupled to the inner shaft and configured to be transitioned from a disengaged configuration to an engaged configuration when the expandable electrode is disposed distal to the outer shaft to indicate the expandable electrode has transitioned to the expanded configuration; and
a second switch operatively coupled to the needle and configured to be transitioned from a disengaged configuration to an engaged configuration when the distal tip of the needle is disposed at least a minimum distance from a distal end of the expandable electrode to indicate the pulsed field ablation can be applied.

20. A method, comprising:
positioning a distal portion of a catheter system near a tissue site, the catheter system including an outer shaft defining a first lumen and an inner shaft disposed within the first lumen of the outer shaft;
disposing a distal end of the inner shaft distal to the outer shaft such that an expandable electrode disposed at the distal end of the inner shaft is transitioned from an unexpanded configuration to an expanded configuration;
extending a distal tip of a needle slidably disposed within a second lumen of the inner shaft distal to a distal end of the expandable electrode to insert the needle into the tissue site, the distal tip of the needle being electrically exposed; and
applying pulsed field ablation to a zone of tissue at the tissue site while the distal tip of the needle is inserted into the tissue site and the expandable electrode is spaced from the tissue site.

21. The method of claim 20, wherein the expandable electrode in the expanded configuration has a conical shape in which a diameter of the expandable electrode increases distally.

22. The method of claim 21, wherein the expandable electrode has a first diameter at a proximal end of the expandable electrode and a second diameter at the distal end of the expandable electrode, and a ratio of the second diameter to the first diameter is at least about 1.1.

23. The method of claim 20, wherein extending the needle distal to the distal end of the expandable electrode includes:
extending the needle relative to the inner shaft such that the distal tip of the needle is disposed up to about 80 mm from the distal end of the expandable electrode.

24. The method of claim 20, wherein the expandable electrode and the distal tip of the needle are configured to be electrically polarized with opposite polarities to apply the pulsed field ablation to the zone of tissue.

25. The method of claim 20, further comprising:
capturing, using an ultrasound transducer coupled to the catheter system, a view of a portion of the catheter system and patient anatomy near the tissue site to confirm placement of the catheter system.

26. The method of claim 20, further comprising:
capturing, using an ultrasound transducer coupled to the catheter system, a view of a portion of the catheter system and patient anatomy near the tissue site to confirm insertion of the needle into the tissue site.

27. The method of claim 20, further comprising:
delivering, while distal tip of the needle is inserted into the tissue site and the expandable electrode is spaced from the tissue site, a conductive fluid to a region surrounding the expandable electrode to establish at least a portion of a conductive path between the expandable electrode and the distal tip of the needle.

28. The method of claim 27, wherein the outer shaft and the inner shaft define an annular space therebetween, the conductive fluid being delivered through the annular space.

29. The method of claim 20, wherein the expandable electrode is configured to automatically transition from the unexpanded configuration to the expanded configuration when the distal end of the inner shaft is disposed distal to the outer shaft.

30. The method of claim 20, further comprising:
detecting a first switch operatively coupled to the inner shaft has transitioned from a disengaged configuration to an engaged configuration to indicate the expandable electrode has transitioned to the expanded configuration;
detecting a second switch operatively coupled to the needle has transitioned from a disengaged configuration to an engaged configuration to indicate the distal tip of the needle is disposed at least a minimum distance from a distal end of the expandable electrode; and
applying, in response to detecting the first switch and the second switch are in the engaged configuration, the pulsed field ablation to the zone of tissue.
